(19) United States
(12) Patent Application Publication
(10) Pub. No.: US 2006/0142220 A1
(43) Pub. Date: Jun. 29, 2006

(54) Protein modification

(76) Inventors: Patrick Van Berkel, Leiden (NL); Maurice Mannesse, Leiden (NL); Frank Pieper, Leiden (NL)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/531,855

(22) PCT Filed: Oct. 17, 2003
(86) PCT No.: PCT/NL03/00703

§ 371 Date: Nov. 2, 2005

(30) Foreign Application Priority Data
Oct. 17, 2002 (EP) 02079328.7

(51) Int. Cl. A61K 48/00; A61K 38/47

(52) U.S. Cl. 514/44; 424/94.61

(57) ABSTRACT

The present invention relates to a method for changing the half-life of a glycosylated compound by the modification of its O-linked carbohydrates. This modification is preferably carried out enzymatically and aimed at extending the half-life of the compound. Both in vivo and in vitro modification protocols may be used.

### FIELD OF THE INVENTION

The present invention relates to the modification of glycosylated compounds, more specifically to the modification of recombinantly produced glycosylated compounds to increase their circulatory lifetime in the blood.

### BACKGROUND OF THE INVENTION

Glycoproteins are a conjugated form of proteins containing one or more covalently bound carbohydrates. Protein-linked carbohydrates may be classified into two groups depending on the nature of the linkage between the glycan and the protein, viz. N-linked carbohydrates which are attached to the free amino group of asparagine residues and O-linked carbohydrates which are linked to the hydroxyl group of threonine and serine residues.

It is well known that the half-life of glycoproteins, i.e. the time by which 50% of a compound has been cleared from the blood circulation, is highly dependent on the composition and structure of its N-linked carbohydrates. For instance, the removal of sialic acid groups from the carbohydrates of glycoproteins will result in rapid clearance of these glycoproteins from circulation (Morell et al. (1971) J. Biol. Chem. 246. 1461), since the desialylated glycoproteins are recognised by various carbohydrate receptors in the body. Examples of such carbohydrate receptors involved in clearance are the asialoglycoprotein receptor and the mannose receptor on liver cells. The same phenomenon is observed in the case of recombinantly produced human proteins, such as human proteins produced in Chinese hamster ovary (CHO) cells or in transgenic animals, which, in general, contain less sialic acids groups than their non-transgenic counterparts.

As to date, it is generally accepted that N-linked carbohydrates dominate the pharmacokinetic properties of a glycoprotein. Therefore, the preferred strategy to improve the half-life of a glycoprotein has been modification of its N-linked carbohydrate groups, through sialylation or removal of terminal galactose residues.

### DETAILED DESCRIPTION

The present invention relates to a method for changing the half-life of a glycosylated compound by the modification of an O-linked carbohydrate. When referring herein to a glycosylated compound, preferably a glycosylated protein or a compound comprising the glycosylated protein is meant. In this context, half-life is defined as the time by which 50% of a compound has been cleared from the blood circulation. In this context “carbohydrate” refers to both monosaccharides and oligosaccharides. As to date, it was thought that half-life was highly dependent on and mainly dominated by the composition and structure of N-linked carbohydrates. The inventors demonstrate that also O-linked carbohydrates may govern the half-lives of glycosylated compounds.

This is of direct relevance to therapeutical application of glycosylated compounds that are administered parenterally, because according to the method of the present invention half-lives may be dramatically prolonged.

The method of the invention may be used to either reduce or increase the half-life of a glycosylated compound which is herein referred to as ‘changing the half-life’. In one embodiment of the invention, the modification at the O-linked carbohydrate is used to extend the half-life of a glycosylated compound. By this modification, the half-life of the modified glycosylated compound is increased by at least 10%, preferably by at least 30%, 50% or 70% as compared to the unmodified compound. Most preferred is that the value of the half-life of the modified glycosylated compound has increased to at least twice, three times or four times the value of the half-life of the unmodified compound.

The modification of the O-linked carbohydrate is preferably carried out enzymatically by using an enzyme preparation. The enzyme preparation may comprise one enzyme or a mixture of enzymes. These enzymes may have a varying degree of purity. They may be purified or substantially pure, but this is not an absolute requirement.

Both in vivo and in vitro modification protocols may be used to modify the O-linked carbohydrates. Examples of in vivo modification include, but are not limited to, modifications that take place in cell culture systems or in transgenic animals or in transgenic bacteria or plants, for example by co-expression of one or more suitable enzymes.

The modification of the O-linked carbohydrates is concentrated on the capping or removal of terminal galactose residues thereby interfering with the binding to receptors involved in clearance and therefore leading to a prolonged circulatory life time in the blood. Suitable enzymes include, but are not limited to, sialyltransferases for capping terminal galactose, such as for example ST3GalIII or ST3GalI or other sialyltransferases as known in the art. Examples of enzymes which are useful for the removal of terminal galactose are galactosidases and endo-acetylgalactosaminidases (O-glycosidase). Galactosidases are capable of removing terminal galactose from either N- or O-linked carbohydrates, whereas endo-acetylgalactosaminidases hydrolyse the covalent linkage between the polypeptide and galactosamine (O-linked to either serines or threonines) of non-sialylated Galβ1,3GalNAc structures. In both cases the number of exposed galactose residues will be reduced and will therefore enhance the circulatory life time of the glycoprotein.

A preferred way of modification of the O-linked carbohydrate group is sialylation. In general, sialylation involves the transfer of sialic acid from a sialic acid donor to a carbohydrate group on a glycosylated compound by the action of a sialyltransferase. This may either take place in vivo (for example by co-expression of the sialyltransferase in the glycoprotein expression system) or in vitro. To date, cytidine-5′-monophospho-N-acetylneuraminic acid (CMP-sialic acid) is commonly used as the sialic acid donor. The sialyltransferase may be recombinantly produced or isolated from a sialyltransferase source. Methods for producing recombinant sialyltransferases have been published, e.g. in U.S. Pat. No. 5,541,083. A preferred example of a sialyltransferase to be used in the method of the invention is ST3Gal III (EC 2.4.99.4), preferably human ST3 Gal I, but sialyltransferases from non-human mammals or bacterial origin may also be used, preferably in combination with ST3Gal III (EC 2.4.99.6). ST3Gal I specifically transfers a sialic acid to the terminal galactose of Galβ1,3GalNAc epitopes which is the core structure of mucin type O-linked carbohydrates, whereas ST3 Gal III is specific for lactosamine units (Galβ1,4GlcNAc) often occurring in complex and hybrid type N-linked carbohydrates. The method described herein may be used to improve the pharmacokinetic properties of any glycosylated compound especially those bearing mucin type O-linked carbohydrates. Sialylation may be performed using known methods, for instance such as described in WO 98/31826.

Alternatively, the circulatory half-life of a glycosylated compound may be extended through modification of its O-linked carbohydrate groups by removing part or all of an O-linked carbohydrate chain. Preferably one or more of the non-sialylated O-linked carbohydrate chains are removed in part or completely. For example, one or more non-sialylated O-linked galactoses may be removed from one or more carbohydrate chains. As described above, removal of one or more O-linked carbohydrates or carbohydrate chains can be done either in vivo or in vitro. In one embodiment for in vivo removal, the nucleotide sequence encoding one or more suitable enzymes, such as for example galactosidases and/or endo-acetylgalactosaminidases, is co-expressed in the same cells as the glycoprotein. The nucleotide sequences encoding suitable enzymes may be derived from any source, such as human, mouse, rat, bacteria and the like, or may be synthesized chemically. In one embodiment for in vitro removal, one or more suitable enzymes are added to the recombinant glycoprotein in vitro.

Any glycosylated compound of which the half-life has to be modified may be used in the method according to the invention. In this way a compound may be obtained of which the plasma circulatory half-life has been reduced or extended, compared to the half-life of the unmodified compound. Preferably, the half-life is reduced or extended by at least 10%, at least 30%, at least 50% or by at least 70%. Most preferably the value of the half-life has decreased with or increased to at least one and a half, twice, three times or four times the value of the half-life of the unmodified compound. The compound may for instance have been obtained after the sialylation of an O-linked carbohydrate or the removal of one or more non-sialylated O-linked carbohydrates. Typically, the non-sialylated O-linked carbohydrate is galactose or Gal(β1-3)GalNAc. These modifications are preferably performed enzymatically, for instance using an enzyme preparation which comprises one or more enzymes. Suitable enzyme preparations include one or more sialyltransferases, one or more galactosidases and one or more endo-acetylgalactosaminidases. These three types of enzymes may be used alternatively. In one embodiment an enzyme preparation comprising sialyltransferases ST3GalIII and ST3GalI is used to obtain a compound according to the invention. In another embodiment, an enzyme preparation comprising endo-α-N-acetylgalactosaminidase is used to obtain the modified compound. The skilled person will understand that two or all three types of enzymes may also be used in combination The compounds of the inventions may be used to prepare pharmaceutical compositions for the treatment of individuals in applications where normally the unmodified counterparts are used. The pharmaceutical composition will typically also comprise a pharmaceutically acceptable carrier and optionally a pharmaceutically acceptable adjuvant.

Preferably, the method is used for recombinantly produced glycoproteins. The method is extremely useful for improving the half-life of a recombinantly produced glycoprotein that is intended to be administered parenterally.

In this context “recombinantly produced glycoproteins” or “recombinant glycoproteins” refers to glycoproteins which are produced by cells which replicate a heterologous nucleic acid, or expresses a peptide or protein encoded by a heterologous nucleic acid. The heterologous nucleic acid typically contains one or more genes which are not found in the native or natural form of the cell or which may be found in such cell but which have been modified or manipulated. The heterologous nucleic acid may be integrated into the genome of the transformed cell. It is understood that the recombinant glycoprotein does not need to comprise a full-length glycoprotein, but may comprise a functional fragment thereof. Also functional variants of naturally occurring glycoproteins are suitable, such as proteins with conservative amino acid substitutions. As used herein, the term “functional” indicates that at least 80%, or at least 85% or 90%, preferably at least 95% of the chemical biological activity of the full-length glycoprotein or of the naturally occurring glycoprotein is retained. Molecular cloning techniques for producing recombinant molecules are known in the art and have been described in several places, for example Sambrook and Russell (2001) Molecular Cloning: A Laboratory Manual, Third Edition, Cold Spring Harbor Laboratory Press, NY. Suitable cells for expression comprise eukaryotic cells, and include mammalian, fungal and insect cells.

In the context of this application, recombinantly produced glycoproteins are preferably produced in mammalian cell culture systems or in transgenic animals, such as in goat, sheep and cattle. Methods for producing in these systems have been described and are known to the person skilled in the art, see for instance WO 97/05771. The glycoprotein may be obtained from these production systems in a manner known per se for isolating and/or purifying recombinantly produced proteins, see generally Scopes, Protein Purification (Springer-Verlag, New York, 1982). In vitro modification may take place during or after isolation or purification. If it is implemented during purification, it has the advantage that modification additives may be removed during downstream processing.

In one embodiment of the invention a modified recombinant glycoprotein is provided. “Modified recombinant glycoprotein” as used herein refers to a recombinant glycoprotein comprising one or more modified O-linked carbohydrates, whereby the blood circulatory half-life of the recombinant glycoprotein is changed, preferably increased to at least 1.5, 2, 3 or 4 times the value of the half-life of the unmodified recombinant glycoprotein. It is noted that recombinant glycoproteins may differ from non-recombinant (natural) glycoproteins in a number of aspects. In particular, the glycosylation pattern of the recombinant glycoprotein may be different from that of the non-recombinant glycoprotein. For example, while the structure of the N-linked glycans of non-recombinant glycoproteins may be complex its recombinant counterpart may contain structures of the high mannose type. A recombinant glycoprotein can therefore be distinguished from a non-recombinant glycoprotein by HPAEC-PAD profiling (=high performance anion-exchange chromatography pulsed amperometric detection), in particular as described in the Examples.

In one embodiment, recombinant human C1 inhibitor (rhC1INH), purified from the milk of transgenic rabbits, is sialylated in vitro by using a mixture of recombinantly produced sialyltransferases. A modified rhC1INH may be used for treating individuals and preparing pharmaceutical compositions, for instance as described in WO 01/57079.

It will be clear to the skilled person that the half-life of a glycosylated compound may be reduced by increasing the number of terminal galactose residues. This may for instance be achieved by treatment with a sialidase, such as for example sialidase EC 3.2.1.18. The half-life of a glycosylated compound may be reduced by at least 10%, preferably by at least 30%, 50% or 70% as compared to the unmodified compound. More preferably, the half-life is decreased to at least 1.5, 2, 3 or 4 times the value of the half-life of the unmodified compound. Preferably, the galactose residues which are present on O-linked carbohydrate chains are involved in this process.

It is clear that the following examples do not limit the invention in any way. Unless stated otherwise in the Examples, all molecular techniques are carried out according to standard protocols as described in Sambrook and Russell (2001) Molecular Cloning. A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK).

### EXAMPLES

### Experimental

Sialic Acid Determination

Sialic acids on rhC1INH samples produced in rabbits were quantified in the following way: sialidase from Arthrobacter ureafaciens was added to rhC1NH and samples were incubated for 1 h at 37° C. The amount of released sialic acid was quantitated determined on HPAEC-PAD after adding 3-deoxy-D-glycero-D-galacto-2-nonulosonic acid (KDN, Toronto research Chemicals) as an internal control.

SDS-PAGE and Inhibitory Activity

Non-reduced and reduced SDS-PAGE was performed using the Novex system as recommended by the manufacturer. Proteins were visualized by silver staining. Inhibitory activity of rhC1INH, either before or after in vitro sialylation, was determined according to a standard procedure with the target protease C1s in the presence of a synthetic chromogenic substrate. After determining the rhC1NH antigen concentration by an ELISA assay, the specific activity in mU/mg protein was calculated.

N-Linked Glycosylation Profiling

N-linked glycosylation profiling was performed according to an in-house method. Briefly, rhC1INH was diluted in 25 mM sodium phosphate, 62 mM EDTA, pH 7.2 containing 5 mg/ml N-octylglucoside and boiled for 2 min. Subsequently, N-glycosidase F was added and samples were incubated for 45 h at 37° C. Samples were rotated for 5 min at 14,000 rpm and supernatant was analyzed on a Carbopac PA-1 column with Carbopac PA-100 guard, pre-equilibrated in 150 mM NaOH. Carbohydrates were eluted with a 0-175 mM sodium acetate gradient in 150 mM NaOH at 1 ml/min in 30 min.

O-Linked Glycosylation Profiling

The O-linked carbohydrates were removed from rhC1INH by O-elimination after the N-linked carbohydrates had been removed from the rhC1INH preparations. Therefore, 200 μg of rhC1INH was treated with N-glycosidase F as described above, with the exception that samples were digested for 17 h instead of 48 h. After deglycosylation, samples were mixed with three volumes of 96% (v/v) ethanol and incubated for 10 min on ice before rotation for 5 min at 15000 rpm at 4° C. Protein pellets were twice dissolved in water and precipitated again with ethanol. After the second wash, pellets were dried in a SpeedVac at room temperature and subsequently dissolved in 100 μl 1.0 M NaBH₄, 50 mM NaOH and incubated for 17 h at 45° C. β-elimination was stopped by the addition of HAc (0.8 M final concentration) on ice. Samples were dried in a SpeedVac and washed three times with 1% HAc in methanol. After the third wash, pellets were dissolved in 100 μl water and samples were loaded on a Biorad AG50WX12 column (1 ml packed beads per sample) pre-equilibrated in water. Columns were eluted with three column volumes of water. Fractions were dried in the SpeedVac and resuspended in 100 μl of water. Samples were loaded on a Carbopac PA-1 column with Carbopac PA-100 guard, preequilibrated in 150 mM NaOH. Carbohydrates were eluted with a 0-250 mM sodium acetate gradient in 150 mM NaOH at 1 ml/min in 30 min.

### Example 1

### In Vitro Sialylation Protocol A

In vitro sialylation was carried out by Neose Technologies, Inc (la Jolla, Calif.) on batch 04100011 recombinant human C1 inhibitor produced in the milk of transgenic rabbits, in 50 mM Tris, 0.15 M NaCl, 0.05% NaN₃, pH 7.2 by the addition of the sialyltransferase ST3Gal III in combination with CMP-Sialic acid. After incubation for 16 h at 32° C. samples were frozen in liquid nitrogen and shipped. Samples were thawed and dialysed in Spectrapor membranes with a cut-off of Mr 25,000 against 10 mM sodium phosphate, 0.15 M NaCl, pH 7.4 (PBS) before applying the assays described in this application.

Determination of the amount of sialic acids on rhC1INH revealed that it had increased from 7 mol sialic acid/mol rhC1INH to 9 mol/mol. In vitro sialylation had no impact on the protease inhibitory activity of the protein and did not cause any degradation or aggregation.

N-linked glycosylation profiling of rhC1NH showed that in vitro sialylation caused a significant increase, i.e. about 7-fold, in the amount of double-sialylated structures. Not all the mono-sialylated structures could be converted into double sialylated structures, suggesting that the remaining structures did not contain acceptor sites for the sialyltransferase(s).

O-linked glycosylation profiling of rhC1INH showed only a minor increase in the amount of sialylated Gal-GalNAc, indicating that only a minor portion of the sialic acids had been incorporated into the O-linked carbohydrates.

### Example 2

### In Vitro Sialylation Protocol B

In vitro sialylation on recombinant human C₁-Inhibitor batch 04i00011 in 50 mM Tris, 0.15 M NaCl, 0.05% NaN₃, pH 7.2 was performed as described in Example 1, but now a mixture of sialyltransferases ST3Gal III and ST3Gal I was used.

Determination of the amount of sialic acids on rhC1INH revealed that it had increased from 7 mol sialic acid per mol rhC1INH to 28 mol/mol.

N-linked glycosylation profiling of rhC1INH showed that in vitro sialylation caused a significant increase, i.e about 7-fold, in the amount of double-sialylated structures. Also in this case, not all mono-sialylated structures could be converted into double-sialylated structures.

The O-linked glycosylation profiling of rhC1INH showed that the majority of the sialic acids had been incorporated into the O-linked carbohydrates, i.e. the amount of mono-sialylated Galβ1,3GalNAc increased approximately 10-fold.

Results are summarised in Table 1 (N-linked glycosylation profiling) and Table 2 (O-linked glycosylation profiling) and clearly show that there is no difference in the N-linked profile of both samples, whereas the O-linked profile showed significant differences. Hence, the most important difference between sample rhC1INH-A and rhC1INH-B is in the degree of sialylation of the O-linked carbohydrates.

TABLE 1
Relative peak areas of the N-linked glycan HPAEC-PAD profile
of in vitro sialylated recombinant human rhC1INH.

| | Relative peak area (%) | | |
|---|---|---|---|
| | rhC1INH | rhC1INH- | rhC1INH- |
| Charge group | 04i00011 | protocol A | protocol B |
| Uncharged | 20 | 17 | 18 |
| Mono-charged | 74 | 49 | 45 |
| Double-charged | 5 | 34 | 37 |

TABLE 2
Relative peak areas of the O-linked glycan HPAEC-PAD
profile of in vitro sialylated rhC1INH.

| | Relative peak area (%) | | | |
|---|---|---|---|---|
| | | | | Plasma |
| | | | | C1 |
| Peak | rhC1INH | rhC1INH-A | rhC1INH-B | Inhibitor |
| Uncharged | 85 | 77 | 34 | 11 |
| Monocharged | 10 | 19 | 59 | 78 |
| Double | 4 | 4 | 7 | 11 |
| charged | | | | |

### Example 3

### Pharmacokinetics of Modified rhC1INH

Rats were anaesthetised by subcutaneous injection of hypnorm/midazolam and the abdomen was opened. The test items, i.e. rhC1INH samples, were injected via the tail vein or the vena cava or the vena penis. At the indicated times, blood samples of approximately 0.2 ml were taken from the inferior vena cava and transferred to eppendorf vials with 10 μl of 0.5 M EDTA in PBS. The samples were centrifuged for 5 min at 3500×g and 100 μl plasma of each sample was stored at −20° C. upon analysis. The plasma samples were analysed by using an ELISA for the detection of rhC1INH. Recombinant human C1INH had a plasma circulatory half-life of 16±3.7 min. whereas rhC1INH-A and rhC1INH-B had a half-life of 25±3 and 75±14 min, respectively. The half-life of rhC1INH-B was similar to what we measured previously for human C1 Inhibitor isolated from human plasma, i.e 75±14 min.

These results clearly show that improvement of half-life was obtained through modification of the O-linked carbohydrates. This improvement may be obtained over the unmodified protein (almost 5 times the original value), but also over the (N-linked) modified protein (3 times the N-linked value). This degree of improvement is a new observation, which has never been reported for O-linked carbohydrates before.

TABLE 5
Half-lives of different C1 INH samples

| | Half-life |
|---|---|
| | (min) |
| rhC1INH | 16 ± 3.7 |
| rhC1INH ST3Gal III | 25 ± 3 |
| rhC1INH ST3Gal III + ST3Gal I | 75 ± 14 |
| plasma C1 Inhibitor | 75 ± 14 |

### Example 4

### In Vitro Modification of rhC1INH O-Linked Carbohydrates by Using Endo-α-N-Acetylgalactosaminidase

Removal of non-sialylated O-glycans from rhC1INH was accomplished by using a commercially available recombinant Endo-α-N-Acetylgalactosaminidase (O-glycosidase, Prozyme, which is specific for the hydrolysis of Galβ1,3GalNAcα-Ser/Thr-structures). To this end, different amounts of O-glycosidase, ranging from 0.125-3.25 mU, were added to 200 μg of rhC1INH in 40 μl of a 20 mM phosphate buffer of pH 5.0. The mixture was incubated overnight at 37° C. after which the protein was precipitated and washed three times with 70% ethanol to remove the released Galβ1,3GalNAc. Subsequently, the samples were subjected to O-glycan profiling by using HPAEC-PAD as described in the experimental section. The chromatograms of unmodified and modified rhC1INH showed that O-glycosidase treatment significantly reduced the amount of Galβ1,3GalNAc on rhC1INH. The Galβ1,3GalNAc peak was reduced to approximately 25% as compared to the unmodified rhC1INH. Moreover, O-glycosidase treatment did not affect the protease inhibitory activity of rhC1INH nor did it cause aggregation of degradation. The reduction in the number of non-sialylated O-glycans in rhC1INH is expected to lead to improved pharmacokinetics of the modified product.

The method described herein may be used to improve the pharmacokinetic properties of any glycosylated compound bearing mucin type O-linked glycans.

1. C1 inhibitor which is characterised in that its plasma circulatory half-life has been changed by modification of an O-linked carbohydrate.

2. C1 inhibitor according to claim 1 which is characterised in that its plasma circulatory half-life has been extended compared to the half-life of unmodified C1 inhibitor.

3. C1 inhibitor according to claim 1 which is characterised in that its plasma circulatory half-life has been reduced compared to the half-life of unmodified C1 inhibitor.

4. C1 inhibitor according to claim 1, which is characterised in that the plasma circulatory half-life of the modified inhibitor has decreased with or increased to at least 1.5, 2, 3 or 4 times the value of the half-life of the-unmodified inhibitor.

5. C1 inhibitor according to claim 1, which is characterised in that the modification comprises sialylation of the O-linked carbohydrate or the removal of one or more non-sialylated O-linked carbohydrates.

6. C1 inhibitor according to claim 5, which is characterised in that the non-sialylated O-linked carbohydrate is galactose or Gal(•1-3)GalNAc.

7. C1 inhibitor according to, which claim 1 is characterised in that the O-linked carbohydrate is modified by incubation with an enzyme preparation which comprises one or more enzymes.

8. C1 inhibitor according to claim 7, which is characterised in that the enzyme preparation comprises one or more sialyltransferases, galactosidases or endo-acetylgalactosaminidases.

9. C1 inhibitor according to claim 8 which is characterised in that the enzyme preparation comprises sialyltransferases ST3Gal III and ST3Gal I, or endo-α-N-acetyl-galactosaminidase.

10. C1 inhibitor according to claim 1, which is characterised in that the modification is an in vitro modification.

11. C1 inhibitor according to claim 1, which is characterised in that the C1 inhibitor is human C1 inhibitor.

12. C1 inhibitor according to claim 1 which is characterised in that the C1 inhibitor is recombinantly produced.

13. A pharmaceutical composition comprising C1 inhibitor according to claim 1.

14-15. (canceled)

16. A method for extending the blood circulatory half-life of a glycoprotein or of a glycoprotein comprising compound, wherein the method comprises removing one or more non sialylated O-linked carbohydrates from the glycoprotein.

17. The method according to claim 16 wherein the non-sialylated carbohydrate is galactose or Gal(β1-3)GalNAc.

18. The method according to claim 16 wherein the carbohydrates are removed by in vitro incubation with an enzyme preparation comprising one or more enzymes.

19. The method according to claim 18, wherein the enzyme preparation comprises galactosidase or endo-acetylgalactosaminidase.

20. The method according to claim 18 wherein the enzyme preparation comprises one or more recombinantly produced enzymes.

21. The method according to claim 16, wherein the carbohydrates are removed by in vivo by expression of a nucleic acid encoding a galactosidase or an endo-acetylgalaotosaminidase.

22. The method according to claim 16, wherein the glycoprotein is C1 inhibitor.
